(19) United States
(12) Patent Application Publication
(10) Pub. No.: US 2015/0320491 A1
(43) Pub. Date: Nov. 12, 2015

(54) BIPOLAR COAGULATION AND CUTTING ELECTRODE

(71) Applicant: OLYMPUS WINTER & IBE GMBH, Hamburg (DE)

(72) Inventors: Andreas HÖRLLE, Berlin (DE); Christopher SPRENGER, Berlin (DE); German KLINK, Berlin (DE)

(21) Appl. No.: 14/441,966

(22) PCT Filed: Oct. 29, 2013
(86) PCT No.: PCT/EP2013/072606

§ 371 Date: May 11, 2015

(30) Foreign Application Priority Data
Nov. 13, 2012 (DE) 10 2012 220 682.5

(51) Int. Cl. A61B 18/14

(52) U.S. Cl. CPC A61B 18/1485; A61B 2018/00607

(57) ABSTRACT

The invention relates to a bipolar electrosurgical instrument with an elongated shaft and with two coagulation electrodes arranged one after another in the longitudinal direction of the shaft and each forming a surface portion of the shaft, said electrodes being insulated electrically from one another by an insulator. At the distal end, the shaft is blunt, particularly rounded off, and has a cutting electrode for electrosurgical cutting at the distal end.

The invention relates to a bipolar electrosurgical instrument with an elongate shaft and with two coagulation electrodes which are arranged on the shaft one behind the other in the longitudinal direction of the shaft and which each form a surface portion of the shaft, said coagulation electrodes being electrically insulated from each other by an insulator.

Electrosurgical instruments of the aforementioned type are known from the prior art and are used, for example, in the electrosurgical coagulation and/or ablation of biological tissue. For this purpose, an RF voltage of different potentials (bipolar) is applied to the electrodes, as a result of which the tissue surrounding the electrodes is heated to the extent that the body's own proteins denature.

The prior art likewise discloses bipolar coagulation instruments with a mechanically cutting/puncturing tip, for example a trocar.

The object of the present invention is to make available an electrosurgical instrument which is versatile in use and safe.

In an electrosurgical instrument of the aforementioned type, the object is achieved by the fact that the distal end of the shaft is blunt, in particular rounded, and the distal end has a cutting electrode, connected rigidly to the shaft, for electrosurgical cutting. The cutting electrode has a much smaller surface area than the coagulation electrodes. The cutting electrode can, for example, have the shape of an electrode pole arranged fixedly on the face of the shaft.

The solution has the advantage that an electrosurgical instrument that permits both coagulation and also cutting can be brought safely to a target location. By applying a bipolar RF cutting voltage (e.g. 2.5 kV) to the cutting electrode and the distal coagulation electrode, the cutting function can be activated by an electric arc for electrosurgical cutting being activated between cutting electrode and distal coagulation electrode. For coagulation, a bipolar RF coagulation voltage (e.g. 300 V) can be applied to the coagulation electrodes.

The invention is based on the recognition that bipolar electrosurgical instruments according to the prior art, which have a mechanically cutting/puncturing tip, cannot be conveyed uncovered through a bronchoscope or a guide sheath to their target location, since it is otherwise highly probable that the guide sheath and/or the work channel of the bronchoscope are damaged by the mechanically cutting/puncturing tip.

Thus, in order to promote the initiation of an electric arc, the cutting electrode can protrude in the distal direction beyond the blunt distal end of the shaft. A length by which the cutting electrode protrudes beyond the blunt distal end is preferably shorter than a radius of at least one of the coagulation electrodes, in particular the distal cutting electrode. The instrument can thus be maneuvered particularly safely within tissue. The cutting electrode is preferably arranged coaxially with respect to the shaft.

In the radial direction, the cutting electrode can have a much smaller cross-sectional dimension than the coagulation electrodes.

To make available a compact instrument, one of the coagulation electrodes can be formed by at least a part of the blunt distal end of the shaft. Preferably, the cutting electrode is electrically insulated from the coagulation electrode lying closest to the distal end of the shaft.

According to an advantageous variant of the invention, the cutting electrode is electrically connected to the proximal coagulation electrode. In this case, the instrument requires only two connections for an RF voltage. For a cutting operation, a bipolar RF cutting voltage (for example provided by an RF generator) can be applied to the two coagulation electrodes. If the cutting electrode is electrically connected to the proximal coagulation electrode, this RF cutting voltage always lies as desired also on the cutting electrode, such that an electric arc forms between distal coagulation electrode and cutting electrode. Advantageously, the proximal coagulation electrode, connected to the cutting electrode, is surrounded by an insulating sleeve, e.g. an insertion catheter, such that the electric field in the cutting or puncturing procedure in the tissue forms between the distal coagulation electrode and the cutting electrode. For a coagulation procedure, an RF coagulation voltage can then be applied to the two coagulation electrodes. It is true that in this case a voltage potential also lies on the cutting electrode. Since an RF coagulation voltage is typically much lower than an RF cutting voltage, no electric arc is initiated between coagulation electrode and cutting electrode.

On the other hand, it is also possible that the cutting electrode and the proximal coagulation electrode are electrically insulated from each other. In this case, the instrument requires three connections for an RF voltage, namely for both coagulation electrodes and the cutting electrode. For a cutting operation, a bipolar RF cutting voltage can then be applied to the distal or the proximal coagulation electrode and the cutting electrode. For a coagulation operation, a bipolar RF coagulation voltage is applied to the distal and proximal coagulation electrodes. For an ablation operation, a bipolar RF ablation voltage can be applied to the distal and proximal coagulation electrodes.

Depending on the use of the instrument, the shaft and/or the coagulation electrodes can be designed to be flexible at least in part. It is likewise conceivable for the shaft and/or the coagulation electrodes to be made flexurally stiff.

The shaft preferably has a cylindrical shape at least in the area of the coagulation electrodes. The instrument as a whole can also be substantially cylindrical, as a result of which it is particularly suitable for use with a bronchoscope and/or guide sheath.

It has proven advantageous if the shaft has a lumen for cooling fluid, which lumen reaches at least as far as one of the coagulation electrodes. Thus, the instrument can be cooled from the inside by a cooling fluid, which favors an operation of the instrument free of interruption.

The invention also relates to an electrosurgical system with an instrument as claimed in one of the preceding claims and with a guide sheath, wherein the guide sheath is designed to enclose the instrument at least in part. The guide sheath is preferably designed such that, in a first position, it receives at least the proximal electrode of the instrument completely within a volume spanned by the guide sheath.

The guide sheath is preferably designed to be electrically insulating.

The invention also relates to an electrosurgical method for operating an electrosurgical system, said method having the steps of:
inserting a guide sheath into a tissue to a position before a target location,
inserting the electrosurgical instrument into the guide sheath to such an extent that the cutting electrode of the instrument comes to lie near the target location, wherein at least the proximal electrode of the instrument remains completely inside a volume spanned by the guide sheath when the instrument is intended for a cutting operation.

The method preferably has the step of:
applying an RF cutting voltage to the proximal coagulation electrode and distal coagulation electrode, wherein the proximal electrode of the instrument remains completely inside a volume spanned by the guide sheath.

The method can have the step of:
applying an RF coagulation voltage to the proximal coagulation electrode and distal coagulation electrode, wherein the instrument is pushed out of the guide sheath at least to such an extent that the tissue to be coagulated can come into contact with both coagulation electrodes.

The invention will now be explained in more detail on the basis of illustrative embodiments. In the drawings:

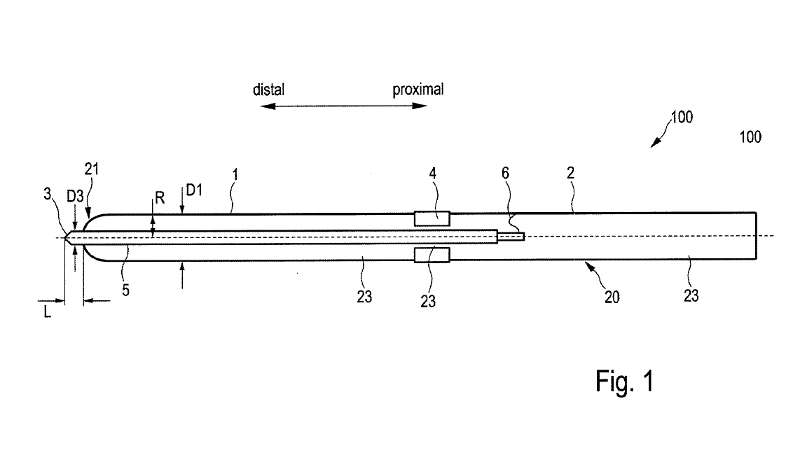

FIG. 1 shows a schematic view of an illustrative embodiment of the instrument according to the invention;

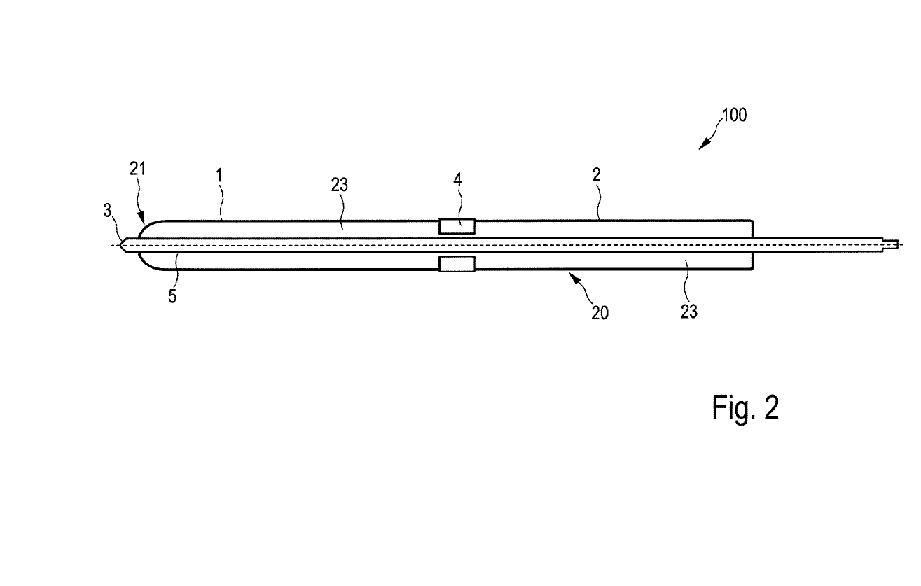

FIG. 2 shows a schematic view of another illustrative embodiment of the instrument according to the invention;

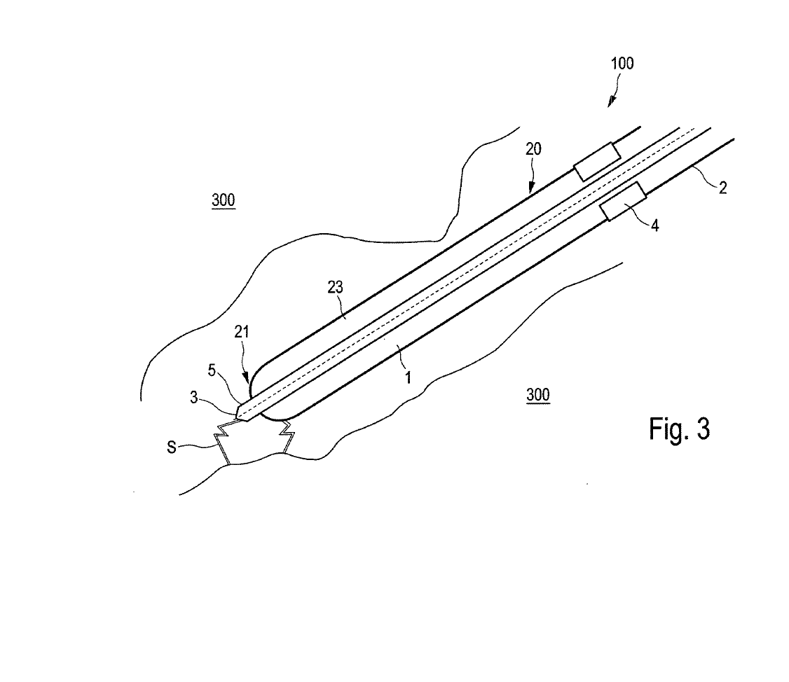

FIG. 3 shows a schematic view of the instrument according to the invention in the cutting operation;

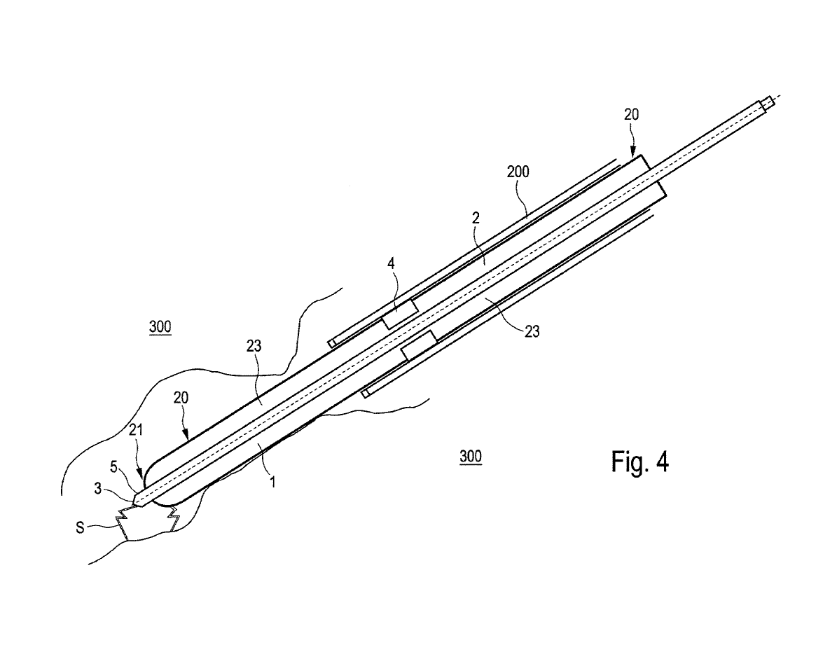

FIG. 4 shows a schematic view of the instrument from FIG. 2 in the cutting operation together with a guide sheath;

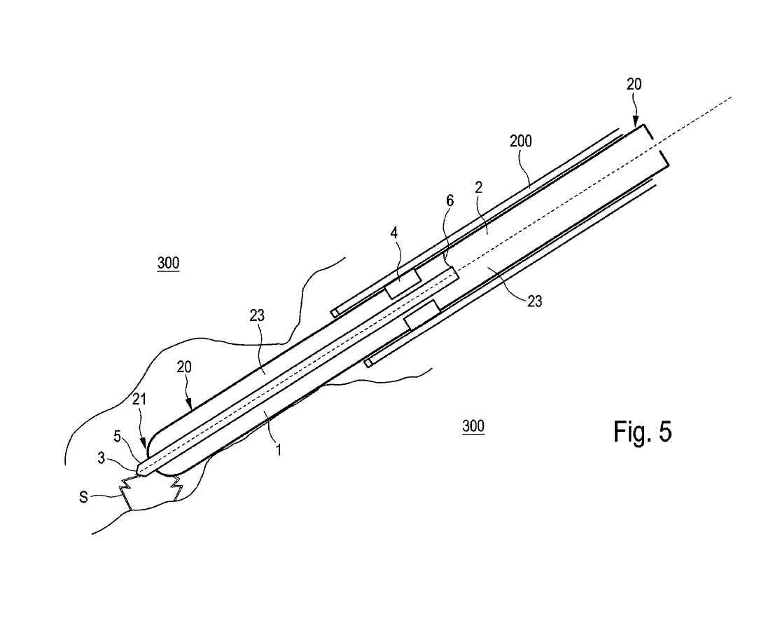

FIG. 5 shows a schematic view of the instrument from FIG. 1 in the cutting operation together with a guide sheath.

A bipolar electrosurgical instrument 100 in FIG. 1 has an elongate, cylindrical shaft 20 and two coagulation electrodes 1, 2 which are arranged on the shaft 20 one behind the other in the longitudinal direction L of the shaft 20. The coagulation electrodes 1, 2 each form a surface portion of the shaft 20 and are electrically insulated from each other by an insulator 4. The insulator 4 is arranged coaxially with respect to the coagulation electrodes 1, 2 and likewise forms a surface portion of the shaft 20. The distal end 21 of the shaft 20 is rounded, wherein the first coagulation electrode 1 forms a part of the rounded distal end 21 of the shaft 20. Overall, except for the rounded distal end 21, the shaft 20 has a cylindrical configuration with a substantially constant circular cross section.

The shaft 20 also has at its distal end 21 a cutting electrode 3 for electrosurgical cutting. It can be seen in FIG. 1 that the cutting electrode 3 permanently protrudes in the distal direction beyond the blunt distal end 21 of the shaft 20 and is arranged coaxially with respect to the shaft 20. A length L by which the cutting electrode 3 protrudes beyond the blunt distal end 21 is shorter than a radius R both of the first coagulation electrode 1 and also of the second coagulation electrode 2.

The surface area of the cutting electrode is much smaller than that of the coagulation electrode, such that a concentration of the electric field takes place on the cutting electrode during operation, which promotes the initiation of an electric arc.

In the radial direction, the cutting electrode 3 has a much smaller cross-sectional dimension (diameter) D3 than the two coagulation electrodes 1, 2 with their cross-sectional dimension (diameter) D1. In the present case, the cutting electrode 3 extends all the way through the first coagulation electrode 1 and is electrically insulated from the first coagulation electrode 1 by an insulating sleeve 5. That is to say, the cutting electrode 3 can be connected to an RF voltage source (not shown) independently of the first (distal) coagulation electrode 1. The cutting electrode 3 extends farther through the insulator 4 into a volume spanned by the second (proximal) coagulation electrode 2. In the present case, the cutting electrode 3 is connected electrically conductively to the second coagulation electrode 2 via an electrical connection element 6, for example a metal wire. The cutting electrode 3 and the proximal coagulation electrode 2 are electrically connected to each other inside the shaft 20. Therefore, if the proximal coagulation electrode 2 is connected to an RF voltage source, the same voltage potential lies both on the cutting electrode 3 and also on the proximal coagulation electrode 2.

As can be seen from FIG. 1, the shaft 20 has a lumen 23 for a cooling fluid. The lumen 23 reaches both to the first coagulation electrode 1 and also to the second coagulation electrode 2 and also to the insulator 4.

The main difference between the instrument 100 shown in FIG. 1 and the instrument 100 shown in FIG. 2 is that the instrument 100 shown in FIG. 2 has a cutting electrode 3 which is electrically insulated both from the first coagulation electrode 1 and also from the second coagulation electrode 2 by means of the insulating sleeve 5. That is to say, an RF voltage potential can be applied to the cutting electrode 3 independently of the first coagulation electrode 1 and of the second coagulation electrode 2. Furthermore, in the instrument 100 shown in FIG. 2, the cutting electrode 3 extends completely through both the first coagulation electrode 1 and also the second coagulation electrode 2. The instrument 100 shown in FIG. 2 is otherwise the same as the one shown in FIG. 1.

The operation of the instrument 100 is explained in more detail below. FIG. 3 shows an instrument 100 that has been inserted into a biological tissue 300. In the present case, there is an RF cutting voltage across the cutting electrode 3 and the coagulation electrode 1. Accordingly, an electric arc S for the electrosurgical cutting on the one hand forms between the cutting electrode 3 and the tissue 300. On the other hand, the circuit between the tissue 300 and the distal coagulation electrode 1 is closed over a large contact area by bodily fluid (blood) between tissue 300 and distal coagulation electrode 1.

An electrosurgical system having an instrument 100 and a guide sheath 200 is shown in FIG. 4. The instrument 100 shown in FIG. 4 corresponds to the one described with reference to FIG. 2, i.e. the cutting electrode 3 is electrically insulated from the proximal coagulation electrode 2. The instrument 100 in the present case is in the cutting operation, i.e. there is an RF cutting voltage across the cutting electrode 3 and the distal coagulation electrode 1 and an electric arc S is initiated between cutting electrode 3 and tissue 300.

The guide sheath 200 in FIG. 4 has a cylindrical shape and encloses the instrument 100 in part. The guide sheath 200 serves to convey the instrument 100 safely to a target location in the tissue 300 and to hold it movably there. For this purpose, the guide sheath 200 is first of all introduced into a tissue or body volume 300, after which the instrument 100 is pushed in.

A further electrosurgical system having an instrument 100 and a guide sheath 200 is shown in FIG. 5. The instrument 100 shown in FIG. 5 corresponds to the one described with reference to FIG. 1, i.e. the cutting electrode 3 is electrically connected to the proximal coagulation electrode 2 via an electrical connection element 6. The instrument 100 in the present case is in the cutting operation, i.e. there is an RF cutting voltage across the proximal coagulation electrode 2 and the distal coagulation electrode 1. Since the cutting electrode 3 is electrically connected to the proximal coagulation electrode 2, the RF cutting voltage also lies across the cutting electrode 3 and the distal coagulation electrode 1, as a result of which an electric arc S is initiated between cutting electrode 3 and tissue 300.

In the present case, the guide sheath 200 is designed to be electrically insulating. In addition to its actual guide function, the guide sheath 200 in this case serves, in the cutting operation, to avoid a short circuit between the two coagulation electrodes 1, 2 via a bodily liquid. In the instrument 100 of FIG. 5, a short circuit between the two coagulation electrodes 1, 2 via a bodily liquid would in principle be conceivable, since there is a comparatively high RF cutting voltage across the coagulation electrodes 1, 2 in the cutting operation.

In a treatment procedure, the guide sheath 200 is first of all inserted into tissue 300 to a position before a target location. The instrument 100 is then pushed into the guide sheath 200 to such an extent that the cutting electrode 3 of the instrument 100 comes to lie near the target location, but the proximal electrode 2 of the instrument 100 remains completely inside a volume spanned by the guide sheath 200. The position of instrument 100 and/or guide sheath 200 may need to be corrected a number of times. At any rate, the guide sheath 200 is designed such that, in a first position (the first position is shown in FIG. 5), at least the proximal electrode 2 of the instrument 100 is received completely within a volume spanned by the guide sheath 200. In a next step, an RF cutting voltage is applied to the two coagulation electrodes 1, 2 for a cutting operation.

For a subsequent coagulation operation, an RF coagulation voltage is applied to the two coagulation electrodes 1, 2, and the instrument 100 is pushed out of the guide sheath 200 at least to such an extent that the tissue 300 to be coagulated can come into contact with both coagulation electrodes 1, 2. Of course, the coagulation operation and the cutting operation can be repeated a number of times. It is also conceivable to perform coagulation prior to a first cutting procedure.

1. A bipolar electrosurgical instrument with an elongate shaft and with two coagulation electrodes which are arranged on the shaft one behind the other in the longitudinal direction of the shaft and which each form a surface portion of the shaft, said coagulation electrodes being electrically insulated from each other by an insulator, wherein the distal end of the shaft is blunt, in particular rounded, and the distal end has a cutting electrode, connected rigidly to the shaft, for electrosurgical cutting.

2. The instrument as claimed in claim 1, wherein one of the coagulation electrodes forms at least a part of the blunt distal end of the shaft.

3. The instrument as claimed in claim 1, wherein the cutting electrode protrudes in the distal direction beyond the blunt distal end.

4. The instrument as claimed in claim 1, wherein the cutting electrode has, in the radial direction, a much smaller cross-sectional dimension than the coagulation electrodes.

5. The instrument as claimed in claim 1, wherein the cutting electrode is electrically insulated from the coagulation electrode, which lies closest to the distal end of the shaft.

6. The instrument as claimed in claim 1, wherein the cutting electrode is electrically connected to the proximal coagulation electrode.

7. The instrument as claimed in claim 1, wherein the shaft and/or the coagulation electrode are designed to be flexible at least in parts.

8. The instrument as claimed in claim 1, wherein a length by which the cutting electrode protrudes beyond the blunt distal end is shorter than a radius of at least one of the coagulation electrodes.

9. The instrument as claimed in claim 1, wherein the shaft has a cylindrical shape at least in the area of the coagulation electrodes.

10. The instrument as claimed in claim 1, wherein the cutting electrode is arranged coaxially with respect to the shaft.

11. The instrument as claimed in claim 1, wherein the shaft has a lumen for a cooling fluid, which lumen reaches as far as at least one of the coagulation electrodes.

12. An electrosurgical system with an instrument as claimed in claim 1 and with a guide sheath, wherein the guide sheath is designed to enclose the instrument at least in parts.

13. An electrosurgical method for operating an electrosurgical instrument and/or system as claimed in claim 1, said method having the steps of:
inserting a guide sheath into a tissue to a position before a target location,
inserting the electrosurgical instrument into the guide sheath to such an extent that the cutting electrode of the instrument comes to lie near the target location,
wherein at least the proximal electrode of the instrument remains completely inside a volume spanned by the guide sheath when the instrument is intended for a cutting operation.
